(19) United States
(12) Plant Patent Application Publication
(10) Pub. No.: US 2017/0094878 P1
(43) Pub. Date: Mar. 30, 2017

(54) Echinacea plant named 'Hilmoooshin'

(50) ;

(71) Applicant: DIONY de BONT, De KWAKEL (NL)

(72) Inventor: DIONY de BONT, De KWAKEL (NL)

(73) Assignee: TAKII EUROPE B.V., De KWAKEL (NL)

(21) Appl. No.: 14/756,656

(22) Filed: Sep. 29, 2015

(51) Int. Cl. A01H 5/00

(52) U.S. Cl. PLT/428

(57) ABSTRACT

A new and distinct cultivar of Echinacea plant named ‘Hilmoooshin’, characterized by its upright plant habit; moderately vigorous growth habit; strong and healthy roots; dark green-colored leaves; strong flowering stems; numerous large inflorescences with bright yellow-colored ray florets; and good garden performance.

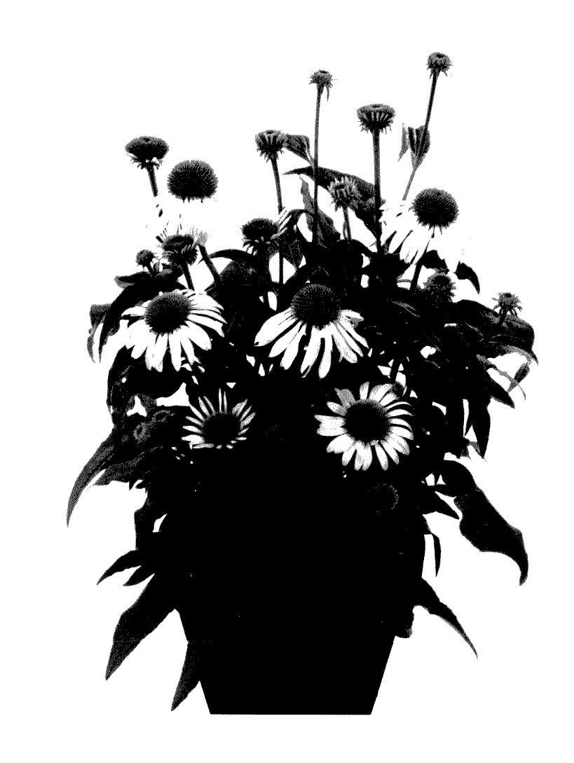

Botanical designation: Echinacea purpurea.

Cultivar denomination: ‘HILMOOOSHIN’.

### BACKGROUND OF THE INVENTION

The present invention relates to a new and distinct cultivar of Echinacea plant, botanically known as Echinacea purpurea, and hereinafter referred to by the name ‘Hilmoooshin’.

The new Echinacea plant is a product of a planned breeding program conducted by the Inventor in De Kwakel, The Netherlands. The objective of the breeding program is to develop new strong and freely flowering Echinacea plants with attractive ray and disc floret coloration.

The new Echinacea plant originated from an open-pollination in June, 2009 in De Kwakel, The Netherlands of a proprietary selection of Echinacea purpurea identified as code designation T 20003 A, not patented, as the female, or seed, parent with an unknown selection of Echinacea purpurea as the male, or pollen, parent. The new Echinacea plant was discovered and selected by the Inventor as a single flowering plant from within the progeny of the stated open-pollination grown in a controlled greenhouse environment in De Kwakel, The Netherlands in July, 2010.

Asexual reproduction of the new Echinacea plant by tissue culture in a controlled environment in De Kwakel, The Netherlands since September, 2010 has shown that the unique features of this new Echinacea plant are stable and reproduced true to type in successive generations.

### SUMMARY OF THE INVENTION

Plants of the new Echinacea have been observed under all possible combinations of environmental conditions and cultural practices. The phenotype may vary somewhat with variations in environmental conditions such as temperature and light intensity, without, however, any variance in genotype.

The following traits have been repeatedly observed and are determined to be the unique characteristics of ‘Hilmoooshin’. These characteristics in combination distinguish ‘Hilmoooshin’ as a new and distinct Echinacea plant:
1. Upright plant habit.
2. Moderately vigorous growth habit.
3. Strong and healthy roots.
4. Dark green-colored leaves.
5. Strong flowering stems.
6. Numerous large inflorescences with bright yellow-colored ray florets.
7. Good garden performance.

Plants of the new Echinacea can be compared to plants of the female parent selection. Plants of the new Echinacea differ from plants of the female parent selection in the following characteristics:
1. Plants of the new Echinacea are not as compact as plants of the female parent selection.
2. Plants of the new Echinacea have stronger roots than plants of the female parent selection.
3. Plants of the new Echinacea have larger inflorescences than plants of the female parent selection.
4. Plants of the new Echinacea and the female parent selection differ in ray floret color as plants of the female parent selection have light orange-colored ray florets. Plants of the new Echinacea can be compared to plants of Echinacea hybrid ‘Cleopatra’, disclosed in U.S. Plant Pat. No. 24,631. In side-by-side comparisons conducted in De Kwakel, The

Netherlands, plants of the new Echinacea differed primarily from plants of ‘Cleopatra’ in the following characteristics:
1. Plants of the new Echinacea had larger leaves than plants of ‘Cleopatra’.
2. Plants of the new Echinacea had larger inflorescences than plants of ‘Cleopatra’.
3. Ray florets of plants of the new Echinacea were lighter yellow in color than ray florets of plants of ‘Cleopatra’.

### BRIEF DESCRIPTION OF THE PHOTOGRAPH

The accompanying photograph illustrates the overall appearance of the new Echinacea plant showing the colors as true as it is reasonably possible to obtain in colored reproductions of this type. Colors in the photograph may differ slightly from the color values cited in the detailed botanical description which accurately describe the colors of the new Echinacea plant.

The photograph is a side perspective view of a typical flowering plant of ‘Hilmoooshin’ grown in a container.

### DETAILED BOTANICAL DESCRIPTION

The aforementioned photograph and following observations and measurements describe plants grown during the spring and early summer in 17-cm containers in a glass-covered greenhouse and “finished” in an outdoor nursery in De Kwakel, The Netherlands under cultural practices typically used in commercial Echinacea production. During the production of the plants, day temperatures ranged from 12° C. to 20° C. and night temperatures ranged from 7° C. to 15° C. Plants used for the photograph and description were 20 weeks old. In the following description, color references are made to The Royal Horticultural Society Colour Chart, 2007 Edition, except where general terms of ordinary dictionary significance are used.
Botanical classification: Echinacea purpurea ‘Hilmoooshin’.
Parentage:
   Female parent.—Proprietary selection of Echinacea purpurea identified as code designation T 20003 A, not patented.
   Male parent.—Unknown selection of Echinacea purpurea, not patented.
Propagation:
   Type.—By tissue culture.
   Time to initiate roots, summer.—About five weeks at temperatures about 20° C. for the first two weeks, then at temperatures about 12° C. to 14° C.
   Time to initiate roots, winter.—About six weeks at temperatures about 20° C. for the first two weeks, then at temperatures about 14° C. to 20° C.
   Time to produce a rooted young plant, summer.—About five weeks at temperatures about 14° C. to 20° C.
   Time to produce a rooted young plant, winter.—About six weeks at temperatures about 18° C.
   Root description.—Medium in thickness, fibrous; strong and healthy; typically white to brown in color, actual color of the roots is dependent on substrate composition, water quality, fertilizer type and formulation, substrate temperature and physiological age of roots.
   Rooting habit.—Moderately freely branching; medium density.
Plant description:
   Plant form and growth habit.—Herbaceous perennial; upright plant habit, narrow inverted triangle; freely branching habit with about nine primary branches developing per plant; moderately vigorous growth habit.
   Plant height.—About 56.8 cm.
   Plant diameter or spread.—About 45.6 cm.
   Lateral branches.—Length: About 43.3 cm. Diameter: About 6 mm. Internode length: About 8 cm. Aspect: Erect to about 15° from vertical. Strength: Strong. Texture: Sparsely pubescent, strigose; rough. Color: Close to 144A.
Leaf description:
   Arrangement.—Alternate, simple.
   Length.—About 13.5 cm.
   Width.—About 4.4 cm.
   Shape.—Narrowly ovate.
   Apex.—Acute.
   Base.—Attenuate.
   Margin.—Entire; slightly undulate.
   Texture, upper and lower surfaces.—Moderately to densely pubescent, strigose; rough.
   Luster, upper surface.—Slightly glossy.
   Luster, lower surface.—Moderately glossy.
   Venation pattern.—Pinnate.
   Color.—Developing leaves, upper and lower surfaces: Close to 144A. Fully expanded leaves, upper surface: Close to between 137A and N137B; venation, close to 145A. Fully expanded leaves, lower surface: Close to 147B; venation, close to 145B.
   Petioles, basal leaves.—Length: About 8.8 cm. Diameter: About 2.5 mm by 3 mm. Strength: Strong, flexible. Texture, upper surface: Smooth, glabrous. Texture, lower surface: Sparsely pubescent, strigose. Color, upper surface: Close to 144B; margins, close to 137A. Color, lower surface: Close to 144A to 144B; margins, close to 137B.
Inflorescence description:
   Appearance.—Terminal rotate single-type inflorescences with ray and disc florets arranged on a capitulum; inflorescences positioned upright above the foliar plane on mostly upright and strong peduncles.
   Flowering habit.—Freely flowering habit with about 27 inflorescences per plant at one time.
   Fragrance.—None detected.
   Time to flower.—Plants begin flowering about 13 to 18 weeks after planting; in the garden, plants flower continuously from mid-June to late September in The Netherlands.
   Inflorescence longevity.—Inflorescences maintain good substance for about three weeks on the plant; inflorescences persistent.
   Inflorescence buds.—Height: About 1.6 cm. Diameter: About 2.9 cm. Shape: Flattened globular. Color: Involucral bracts, close to 146A and 138B; ray florets, close to 154C to 154D.
   Inflorescence size.—Diameter: About 9.8 cm. Depth (height): About 3.2 cm. Disc diameter: About 3.7 cm. Receptacle height: About 1.1 cm. Receptacle diameter: About 1.3 cm. Receptacle color: Close to 155A.
   Ray florets.—Quantity and arrangement: About 24 arranged in a single whorl at the base of the receptacle. Length: About 4.7 cm. Width: About 8 mm. Shape: Oblanceolate. Apex: Emarginate to praemorse. Base: Cuneate. Margin: Entire. Texture, upper surface: Smooth, glabrous; velvety. Texture, lower surface: Smooth, glabrous. Luster, upper surface: Matte. Luster, lower surface: Slightly glossy. Aspect: Horizontal to drooping, about 10° from horizontal. Color: When opening, upper surface: Close to 11A. When opening, lower surface: Close to 10C. Fully opened, upper surface: Close to 13A; color does not change with development. Fully opened, lower surface: Close to 14C; color does not change with development.
   Disc florets.—Length: About 1 cm. Diameter: About 0.5 mm. Shape: Tubular, apices acute. Number of disc florets per inflorescence: About 600. Texture, inner and outer surfaces: Smooth, glabrous. Luster, inner and outer surfaces: Glossy. Color, immature, inner and outer surfaces: Apex: Close to 143A. Mid-section and base: Close to 145C. Color, mature, inner and outer surfaces: Apex: Close to 143A. Mid-section and base: Close to between 143C and 144A.
   Receptacle spines.—Quantity: One per disc floret. Shape: Acicular. Apex: Narrowly acute. Base: Attenuate. Texture: Smooth, glabrous. Luster: Glossy. Color: Apex: Close to 17C. Mid-section: Close to 143A. Base: Close to 145D.
   Involucral bracts.—Quantity per inflorescence: About 64 arranged in about four whorls. Length: About 1.1 cm. Width: About 3 mm. Shape: Ovate to narrowly ovate. Apex: Acute. Base: Cuneate. Margin: Entire. Texture, upper surface: Smooth, glabrous. Texture, lower surface: Moderately pubescent. Color, upper surface: Close to 143C. Color, lower surface: Close to 138B.
   Peduncles.—Length: About 6.7 cm. Diameter: About 5 mm. Strength: Strong. Aspect: Upright. Texture: Moderately pubescent, strigose. Color: Close to 144B to 144C.
   Reproductive organs.—Androecium (present on ray and disc florets): Quantity per floret: About five. Filament length: About 3 mm. Filament color: Close to 150D. Anther shape: Narrowly oblong. Anther length: About 2.5 mm. Anther color: Close to 200B. Pollen amount: Scarce to moderate. Pollen color: Close to 17B. Gynoecium (present only on disc florets): Quantity per floret: One. Pistil length: About 7 mm. Stigma shape: Decurrent, unequal. Stigma color: Close to 153D. Style length: About 5.5 mm. Style color: Close to 145C. Ovary color: Close to 145D. Seeds and fruits: Seed and fruit development have not been observed on plants of the new Echinacea.
Disease & pest resistance: Plants of the new Echinacea have not been shown to be resistant to pathogens and pests common to Echinacea.
Garden performance: Plants of the new Echinacea have exhibited good garden performance and to tolerate rain and wind. Plants of the new Echinacea have been observed to tolerate high temperatures of about 35° C. and to be hardy to USDA Hardiness Zones 3 to 4.

It is claimed:

1. A new and distinct Echinacea plant named ‘Hilmoooshin’ as illustrated and described.
